(19) United States
(12) Patent Application Publication
(10) Pub. No.: US 2014/0335905 A1
(43) Pub. Date: Nov. 13, 2014

(54) Portable device for measuring blood alcohol level by using a mobile device susch as a phone, tablet or laptop

(71) Applicant: Gurpal Singh Bhoot, San Jose, CA (US)

(72) Inventor: Gurpal Singh Bhoot, San Jose, CA (US)

(73) Assignee: Swaive Corporation, San Jose, CA (US)

(21) Appl. No.: 13/986,483

(22) Filed: May 7, 2013

(51) Int. Cl. G01N 33/497; H04W 4/12

(52) U.S. Cl. CPC G01N 33/4972; H04W 4/12
USPC 455/466; 73/23.3; 702/24

(57) ABSTRACT

The portable breathalyzer accessory combines a blood alcohol content measuring tool with a mobile device to measure, store, and send the blood alcohol content of the user. The portable breathalyzer device attaches to a mobile device, wired or wirelessly, to display the blood alcohol content of the user, which is measured through the portable breathalyzer attachment. The blood alcohol content is displayed as a percentage on the mobile device through the corresponding software application. The software application shows the user their blood alcohol content in comparison with the local blood alcohol content limit, which is found by determining the user's location through the GPS technology of the mobile device.

### CROSS-REFERENCE TO RELATED APPLICATIONS

This application claims the benefit of PPA Ser. No. US 61/648,455 filed 2012 May 17 by the present inventors, which is incorporated by reference.

### BACKGROUND OF THE INVENTION

The combination of alcohol intoxication and operation of a vehicle has resulted in a number of incidents over the years and continues to be an issue. The concept of a breathalyzer is anything but new, and was first invented with the intention of reducing the number of drivers under the influence. The presence of breathalyzers in today's society has allowed for law officials to patrol the roads looking for suspected drivers under the influence of alcohol, who are in a position to possibly cause traffic incidents. Even though police officials have removed numerous intoxicated drivers from our streets, there is too much road to be covered all at once. However, police-grade breathalyzers are not the only ones available, as there are many consumer models available for purchase. Yet, there doesn't seem to be enough breathalyzer users, which may be down to a couple of reasons.

One of the reasons why people are not buying and using breathalyzers might be because of the awkward shape, weight and usability of existing breathalyzers. Many of the more accurate breathalyzers available for the average consumer are bigger in size due to the components required. Also, the more accurate the sensor, the more power required to operate the breathalyzer. The larger power requirements need multiple batteries, which in turn tend to make the product much heavier and bigger.

Another reason why consumers may not be using breathalyzers is the perceived accuracy and re-calibration required. The accuracy of many alcohol sensors is dependent on temperature, humidity and the time of operation. Most of the consumer breathalyzers available on the market require the customer to return or send in their breathalyzer to a factory for re-calibration; other companies will replace the customer's existing breathalyzers with newly calibrated devices, which tend to take a couple business days to be shipped. This method is highly inconvenient towards the customer. The invention stated in U.S. Pat. No. 8,224,608, to Son, et al. is a self-calibrating breathalyzer that relies on some of the customer's physical attributes to re-calibrate the device. One of the factors considered during this re-calibration process is the user's metabolism rate and also includes data related to the amount of alcohol consumed; however such characteristics are very dependent on the user and might cause inaccurate results for a friend or individual who is not the customer. The ideal breathalyzer is calibrated specific to the device's components, such as the blood alcohol sensor.

The term smartphone entails electronic devices used as a cellular phone with the capability of running software applications, such as games, utilities, web browsers, etc. The first real smartphone with these capabilities (running and loading applications) was the first generation iPhone, introduced in 2007. Since then, other cell phone companies have begun designing their own versions of smartphones.

A new market using the capabilities of the smartphone has emerged over the last couple of years and continues to grow exponentially. Mobile health is a category containing such hardware devices, more specifically medical devices, which are connected to the smartphone and combined with an application to build a whole new product. This includes products such as the iHealth BP3 Blood Pressure Monitor and the Withings Smart Body Analyzer.

Accordingly, the claimed invention proceeds upon the desirability of providing significant benefits to the user by reducing the size, cost and power consumption, while making the breathalyzer easier to use and maintaining accurate blood alcohol content readings.

### SUMMARY OF THE INVENTION

The invention is a portable breathalyzer device that can measure, store and send the blood alcohol content of the user. The invention connects or plugs directly to a mobile device such as a cell phone, tablet or laptop PC. The invention uses the processing capability of the mobile device to calculate and display the blood alcohol content, store the results, or transmit the results using email or text messaging directly from the mobile device.

### DRAWINGS

The following detailed descriptions, given by way of example, and not intended to limit the present invention solely thereto, will be best understood with the accompanying figures.

FIG. 1 is a block diagram of the portable breathalyzer device in accordance with an exemplary embodiment of the claimed invention;

FIG. 2 is a diagram illustrating the portable breathalyzer device connecting to different standard straws with an exemplary embodiment of the claimed invention;

FIG. 3 is a diagram showing a picture or movie taken of the user blowing in to the portable device with an exemplary embodiment of the claimed invention;

### DETAILED DESCRIPTION

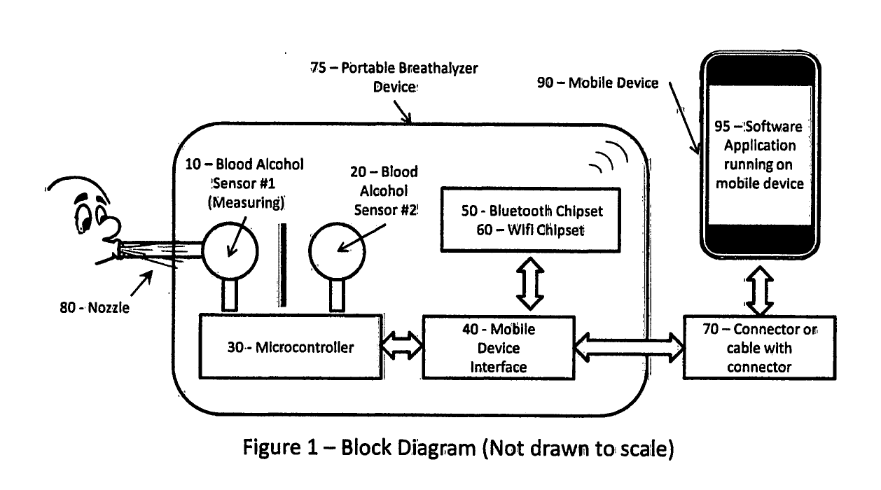

As shown in FIG. 1, in accordance with an exemplary embodiment of the claimed invention, the invention consists of two main embodiments: a portable breathalyzer device 75 and a software application 95. The portable breathalyzer device 75 contains the electronic components required to collect the amount of alcohol present in a breath sample. The data received from the portable breathalyzer device 75 is transmitted to the software application 95 loaded on the mobile device 90. The user interface on the application displays the calculated blood alcohol content of the user.

In accordance with an exemplary embodiment of the claimed invention, the portable breathalyzer device 75 consists of a blood alcohol sensor 10, microcontroller 30 and a mobile device interface 40. The interface between the mobile device 90 and the portable breathalyzer device 75 can be a wired or a wireless interface such as Bluetooth 50 or Wifi 60. If the wired interface is implemented, the portable breathalyzer device 75 is attached to the mobile device 90 through a connector or cable with connector 70. The connector 70 is specific to the mobile device 90 that the corresponding software application 95 is loaded on. Also, the connector 70 allows for the portable breathalyzer device 75 to draw power from the mobile device 90. Therefore, the portable breathalyzer device 75 runs without the need for batteries or any external power. The wireless interface, through the Bluetooth chipset 50 or the WiFi chipset, requires batteries for use. There is also a second blood alcohol sensor 20 allowing for an accurate way to compensate for any effects of temperature or humidity.

The process of determining the user's blood alcohol content percentage begins by loading the software application 95 on the mobile device 90. This can be done in one of two ways: (1) selecting the software application 95 from the list of applications on the mobile device 90, or (2) connecting the portable breathalyzer device 75 to the mobile device 90, which will prompt the user to install (if not already installed) and launch the application automatically. In order to continue or start the testing, the portable breathalyzer device 75 must be connected to the mobile device 90. If the portable breathalyzer device 75 is disconnected at any time during the test, the software application 95 will stop the current test and inform the user to connect the portable breathalyzer device 75. When instructed, the user will blow into the portable breathalyzer device 75, specifically the nozzle 80, for the amount of time allotted by the software application 95. Once the time elapses, the software application 95 will display the results of the test.

The software application 95 contains a feature to determine whether the test was performed correctly. Upon launching the software application 95, the user will press the start button to begin the measurement. After starting the test, the user must blow into the nozzle 80 of the portable breathalyzer device 75 for the specified amount of time, which is shown on the software application 95. The software application 95 can determine if the user is blowing correctly or even blowing at all by evaluating the status of the blood alcohol sensor 10. If the test is begin taken incorrectly, the software application 95 will then restart the test and inform the user accordingly.

The software application 95 contains a feature to vocally instruct the user throughout the testing process. After the test has been initiated, the software application 95 will countdown the remaining time left on the test vocally and also express the results in the same manner. The voice instructions can be turned off by the user.

The software application 95 contains a feature to collect and inform the user of the local blood alcohol content percentage (BAC %) limit. Using the GPS technology implemented in the mobile device 90, the software application 95 will determine the location of the user and determine the local BAC % driving limit. After completing the test, the user will be able to compare their BAC % results with those of the local BAC % driving limit

The software application 95 contains an algorithm to accurately calibrate the portable breathalyzer device 95 without the need for special calibration equipment. The user has the ability to calibrate the portable breathalyzer device 95 at any time. The calibration process consists of the user pressing a calibration icon which starts the calibration process. The software application 95 provides text and voice instructions to the user during the entire calibration process. The calibration process consists of determining a reference value from the blood alcohol sensor 10 while no breath is blown in to the nozzle 80. This reference value is used to determine all future BAC % readings. The calibration algorithm also contains an option to compensate for temperature and humidity. The temperature and humidity is determined by using the sensors on the mobile device 90 or using GPS to determine the geographic location of the user allowing the software application 95 to look up the local weather. The calibration algorithm uses the temperature and humidity to calculate the users BAC %.

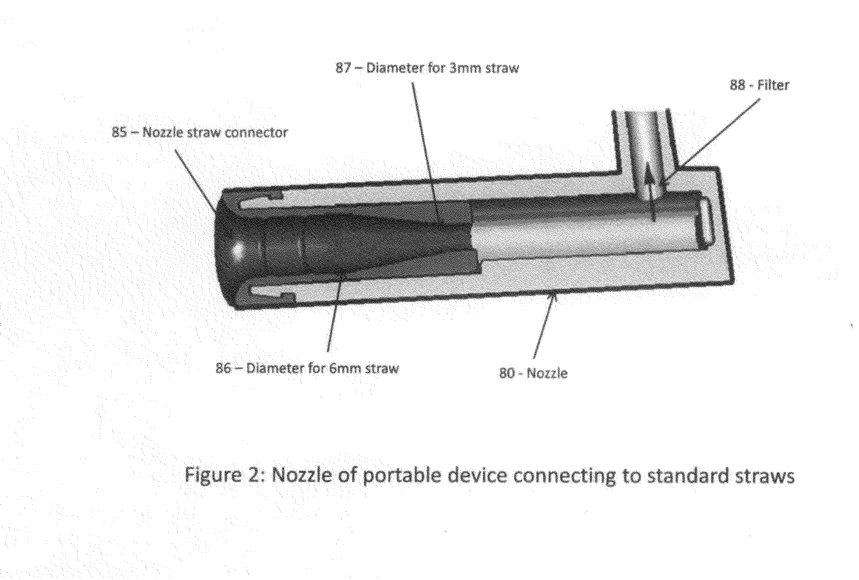

Referring to FIG. 2, the nozzle 80 on the portable breathalyzer device 75 allows for the blood alcohol sensor 10 to be exposed to the right amount of breath sample. The nozzle 80 contains a straw connector 85 which allows for standard sized drinking straws to attach to the nozzle 80. The nozzle straw connector 85 can accommodate standard straws of 6 mm diameter 86 and 3 mm diameter 87. The presence of a straw in this alcohol measuring system allows for multiple users to share the portable breathalyzer device 75 hygienically. The nozzle 80 is designed to minimize and reduce the amount of liquid, spit in the breath, which can travel to the blood alcohol sensor 10 through the filter 88. The material of the filter 10, along with the angle to which the breath sample must travel, allows for the blood alcohol sensor 10 to be exposed only to the air from the breath sample, resulting in ideal testing conditions.

As illustrated in FIG. 1, the portable breathalyzer device 75 also contains a second blood alcohol sensor 20 to provide more accurate results. Blood alcohol sensor#2 20 allows for a low cost method to compensate for effects of temperate and humidity. The blood alcohol sensor#2 20 always provides a value of zero BAC %, as it isn't exposed to the breath sample of the user. The measuring blood alcohol sensor#1 10 provides a BAC % value of the user, as it is exposed directly to the breath sample of the user. The actual BAC % value is determined by the difference between blood alcohol sensor #1 10 and blood alcohol sensor #2 20. This method compensates for any sensitivity to temperature and humidity.

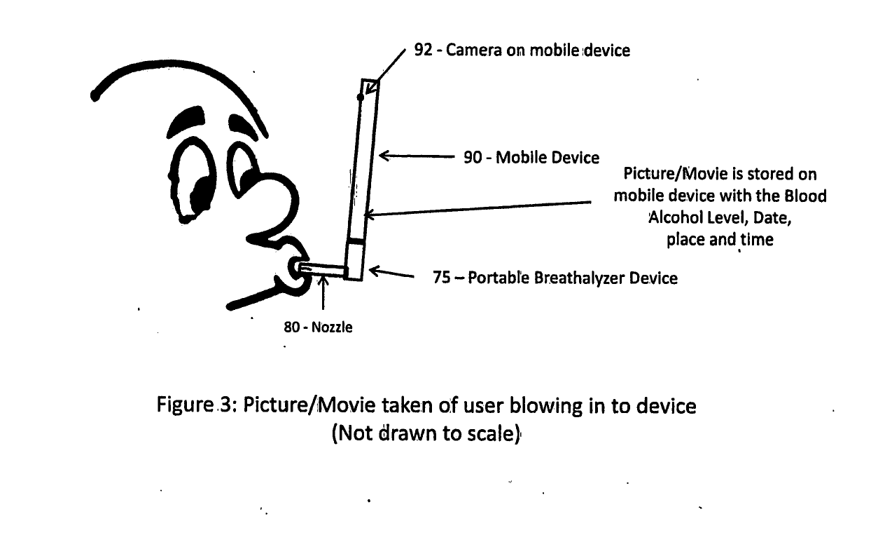

The portable breathalyzer device 75 in conjunction with the software application 95 on the mobile device 90 can be used to take a video or picture of the user during a BAC % test as shown in FIG. 3. During the test, the software application 95 takes a picture or video, using the camera on the mobile device 92, of the user and stores the resulting picture or video along with a time stamp (date, location and BAC%) on the mobile device 90. The stored picture or video can be sent electronically using software application 95 and the capabilities of the mobile device 90.

### Conclusion

Accordingly, the reader will see an invention that takes advantage of the capabilities of mobile devices to display an accurate blood alcohol content percentage (BAC %) reading. The invention combines the portable breathalyzer device with the software application to produce a blood alcohol measuring system unique to the current market of breathalyzer devices. Furthermore, the portable blood alcohol level measuring device has the additional advantages in that:
it allows for usage without the need of external power by consuming the required power from the mobile device;
it displays the local blood alcohol content (BAC %) limit, using the GPS technological ability of the mobile device, which allows the user to compare their results;
it provides an auto-calibration feature which accommodates for the aging of the sensor to provide accurate results.

Although the description above contains many specificities, these should be construed as limiting the scope of the embodiments but as merely providing illustrations of some of several embodiments. For example, the capabilities of the mobile device, containing the software application, include picture or video capturing technology which can be used to validate the identity of the user.

Thus the scope of the embodiments should be determined by the appended claims and their legal equivalents, rather than by the examples given.

I claim:

1. A system for displaying the breath alcohol content of a person's breath sample, comprising:
a. a portable breathalyzer device for analyzing, converting and transmitting said person's breath sample into blood alcohol content percentage, and
b. a software application on a mobile device for displaying said blood alcohol content of said person's breath sample.

2. The system of claim 1, wherein the mobile device is one of the following: a cell phone, a smartphone, a tablet, or a personal computer.

3. The system of claim 1, wherein the portable breathalyzer device connects to a mobile device through a wireless signal, such as wireless local area network, Bluetooth, or cellular data service.

4. The system of claim 1, wherein the portable breathalyzer device is attached to a mobile device through a connector specific to said mobile device.

5. The system of claim 1, wherein the portable breathalyzer device is powered from the mobile device.

6. The system of claim 1, wherein the portable breathalyzer device is powered from an external power source, such as batteries.

7. The system of claim 1, wherein the portable breathalyzer device comprises of:
a. an alcohol sensor converting said amount of alcohol in said person's breath sample into a readable analog voltage, and
b. an electronic component to read and convert said analog voltage into a blood alcohol content percentage.

8. The portable breathalyzer device of claim 7, further comprising a filter to reduce the moisture in the person's breath sample, allowing the conversion results of the alcohol sensor to have greater accuracy and reduce the ability of water damage to said alcohol sensor.

9. The portable breathalyzer device of claim 7, wherein said portable breathalyzer device connects to standard sized straws, allowing for multiple persons to use said portable breathalyzer device hygienically.

10. The system of claim 1, wherein the software application determines the local blood alcohol content limit, using GPS (Global Positioning System) location technology, and compares said blood alcohol content limit to the blood alcohol content of the person.

11. The system of claim 1, wherein the software application contains a user interface displaying operational text messages guiding the user through the test and use process.

12. The system of claim 1, wherein the software application contains a user interface providing audible operational messages guiding the user through the test and use process.

13. The system of claim 1, wherein the software application displays and charts the history of recent test results.

14. The system of claim 1, wherein the software application captures an image or video, using the image capturing technology of the mobile device, of the person using the portable breathalyzer device for the purpose of identifying said person and to electronically send, through email and text messaging, the user's blood alcohol content percentage with said captured image or video to a different person.

15. The system of claim 1, wherein the software application calibrates the accuracy reading of the alcohol sensor, by means of updating the algorithm used in said software application to compensate for effects of temperature and humidity.

16. The system of claim 15, wherein the temperature and humidity data is obtained from using sensors in the mobile device.

17. The system of claim 15, wherein the temperature and humidity data is obtained from GPS location services to determine local weather conditions.
